(19) United States
(12) Patent Application Publication
(10) Pub. No.: US 2015/0148415 A1
(43) Pub. Date: May 28, 2015

(54) DERIVATIZED DENDRIMER WITH LOW CITOTOXICITY FOR IN VIVO, EX VIVO, IN VITRO OR IN SITU CHELATION OF HEAVY METALS OR ACTINIDES

(71) Applicants: Fundacion Fraunhofer Chile Research, Las Condes (CL); Universidad De Talca, Talca (CL)

(72) Inventors: Leonardo SILVA SANTOS, Talca (CL); Rainer FISCHER, Aachen (DE); Luis GUZMAN, Talca (CL); Fabiane M. NACHTIGALL, Talca (CL)

(21) Appl. No.: 14/087,727

(22) Filed: Nov. 22, 2013

(51) Int. Cl. A61K 47/48; A61K 31/16

(52) U.S. Cl. CPC A61K 47/48023; A61K 31/16
USPC 514/517; 558/50; 514/478; 560/165; 514/489; 560/160; 435/2

(57) ABSTRACT

The present invention considers derivatized nanomolecules with proven effectiveness to bind to actinides, more specifically uranium, during in vivo, ex vivo, in vitro or in situ assays. When assayed in vivo, the invention showed a reduction in at least kidney damage due to exposition to uranium.

### TECHNICAL FIELD

The present invention is related to derivatized nanomolecules with proven effectiveness for in vivo, ex vivo, in vitro, or in situ chelation heavy metals or actinides, more specifically uranium or depleted uranium.

### BACKGROUND

Metals are an integral part of many structural and functional components in the body, and the critical role of metals in physiological and pathological processes has always been of interest to researchers.

Uranium is a naturally abundant actinide on Earth and is heavily used in many chemical forms in civilian and military industries. Possible accidental exposure to uranium dust or spatters during the process from mining to industrial application and waste disposal is a matter of concern. Whatever its route of entry into the body, uranium reaches the blood and is partly stored in target organs such as bones and kidneys. Uranium is nephrotoxic, in both human and animal models, and its effects have been widely described. (Taulan et al., 2006)

Depleted Uranium (DU) has been used for counterweights in airplanes and missiles, as radiation shielding, and in inertial guidance devices. Due to its density and ability to undergo phase transition, depleted uranium is also used for anti-tank armor penetrators and as tank armor. The use of DU munitions in military operations has increased the potential exposure of military and civilian personnel to uranium. (May et al., 2004, Barber et al., 2007)

Exposure can occur from handling materials made of DU, inhaling or ingesting dust produced by the firing and impact of DU rounds, or wound contamination. Deployment in combat areas elevated levels of urinary uranium but the observed levels were not outside the normal range. (May et al., 2004)

While studies indicate that most toxicity associated with depleted uranium is due to chemical toxicity and not radioactivity, the health consequences of depleted uranium exposure remain unclear (Hartmann et al., 2000, Barber et al., 2007).

Currently, no known treatments exist for effective removal or treatment of uranium exposure in vivo. The World Health Organization (WHO, “Guidance on Exposure to Depleted Uranium For Medical Officers and Programme Administrators”, Prepared in collaboration with United Nations Joint Medical Staff, 2001) indicate that no known specific treatment for uranium exposure, and that the case of acute exposure should be handled as any heavy metal incorporation, focusing on observed symptoms, and recommending a particular treatment if tubolopathy is diagnosed.

Of the known treatments, metal chelators are one of the available options. The available therapies for metal overload consider using metal chelators such as deferoxamine (DFO, N′-[5-(acetyl-hydroxy-amino)pentyl]-N-[5-[3-(5-aminopentyl-hydroxy-carbamoyl)propanoylamino]pentyl]-N-hydroxy-butane diamide). DFO results effective as metal chelator; nevertheless, it cannot be administered orally and has a very short half-life in serum. Other metal chelators have been developed for clinical use, but serious side effects, such as agranulocytosis (deferiprone, Ferriprox™), renal and liver toxicity (deferesirox, Exjade™) have been found when applied in vivo.

Although, the concept of chelation is based on simple coordination chemistry; evolution of an ideal chelator and chelation therapy that completely removes specific toxic metal from desired site in the body involves an integrated drug design approach (Flora and Pachauri, 2010).

Dendrimers are highly branched, perfectly monodisperse macromolecules with a precisely controlled chemical structure that were first synthesized by Tomalia et al. (Tomalia et al., 1985) and Newkome et al. (Newkome G R. et al., 1985). Specific properties of dendrimers have attracted great interest in terms of exploring their potential in biomedical applications including as drug carriers (Boas and Heegaard, 2004), vectors for gene transfection (Dutta et al.), and as MRI agents (Bumb et al.). In addition, they have contributed significantly to the fields of host-guest chemistry (Pittelkow et al., 2005), and metal complexation (Crooks et al., 2001). Dendrimer architecture, based on multiplied branches, offers advantages including narrow polydispersity, low viscosity compared with equivalent molecular weight linear polymers, and a high density of surface functionalities (Mansfield and Klushin, 1992) Polyamido-amine (PAMAM), poly-L-lysine (PLL), and poly(propylene-imine) (PPI) dendrimers are commercially available and have been widely investigated from a biomedical view point.

The possibility of attaching functional groups such as primary amines, carboxylates, hydroxyl, etc., to dendritic macromolecules is one of the attractive features of dendrimer nanotechnology, thus, dendrimer terminal group can be tuned to develop high capacity and selective dendritic ligands that are capable of trapping the uranyl ion in appropriate media for biological applications.

### PRIOR ART

US2013225645 describe oral formulations based on desazadesferrithiocin polyether (DADFT-PE) analogues that can be administered orally, and that are focused on the treatment of metal overload. One of the metals mentioned in the document for which treatment is intended, is uranium, although no results on uranium chelation or removal are provided, limiting the working invention to iron overload.

WO2006096199 describes the use of compositions and methods for general water treatment, and in a particular disclosed case, for the removal of uranium. The compounds used for water filtration correspond to dendrimers, nevertheless, no modification on the dendrimers is described.

Ilaiyaraja et al (2013) describe a PAMAM G3 dendrimer functionalized with styrene divinylbenzene, used specifically for removal of uranium from aqueous solutions. This cannot be comparable to the present invention, since divinylbenzene has been sindicated as potential carcinogen, and thus, would not be advisable to use as direct in vivo treatment, as the focus of the present invention is.

To the best of the knowledge of the inventors, no similar publications have been done referred to in vivo treatment with an approach similar to the one described in the present document, i.e. treating heavy metal in vivo using a compound based on a functionalized dendrimer.

### BRIEF DESCRIPTION OF DRAWINGS

FIG. 1. Example of the MALDI-TOF spectra obtained of each dendrimer synthesized. A) Arg-Tos coupled to a G4 dendrimer showing 16 coupled molecules and B) Lys-Cbz coupled to a G4 dendrimer showing 39 coupled molecules.

FIG. 2. Fractional binding (% of biding affinity) of the different dendrimers synthesized for uranyl (G4, G5, G4-Arg-Tos, G4-Lys-Cbz, G4-Lys-Fmoc-Cbz, G5-Cou).

FIG. 3. Percent hemolysis of red blood cells subjected to a concentration of 4 μM of the different dendrimers studied (control: Triton X-100, G4, G5, G4-FA, G4-Arg-Tos, G4-Lys-Cbz, G4-Lys-Fmoc-Cbz, G5-Cou).

FIG. 4. Optical microscopy (400×) of different dendrimers incubated at a concentration of 4 μM, with 2% (v/v) of washed Red Blood Cells (RBCs).

FIG. 5. Effectiveness of dendrimers G4-Lys-Fmoc-Cbz and G5-Cou on the concentrations of creatinine (A) and LDH (B). ** p<0.001 compared to saline control. + p<0.05 ++ p<0.001 compared to group UO₂⁻.

FIG. 6. Kidney hystological cut, obtained from different test groups, showing: yellow arrows: necrosis; blue arrows: eosinophilia; red arrows: thyroidization; green arrows: vesiculation.

### DEFINITIONS

The following definitions are provided in order to provide a readily understandable description of the invention.

As used herein, the term “approximately” or “about,” as applied to one or more values of interest, refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “carrier” and “diluent” refers to a safe and non-toxic compound or compounds for administration to a human, useful for the preparation of a pharmaceutical formulation.

As used herein, the term “chelation” means to coordinate (as in a metal ion) with and inactivate.

As used herein, the terms “dosage form” and “unit dosage form” refer to a physically discrete unit of a therapeutic agent for the patient to be treated.

As used herein “dosing regimen” (or “therapeutic regimen”), is a set of unit doses that are to be administered individually to a subject.

As used herein, the term “excipient” refers to any inert substance added to a drug and/or formulation for the purposes of improving its physical qualities (i.e. consistency), pharmacokinetic properties (i.e. bioavailability), pharmacodynamic properties and combinations thereof.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal.

As used herein, the terms “subject”, “individual”, or “patient” refers to a human or any non-human animal (e.g., mouse, rat, rabbit, dog, cat, cattle, swine, sheep, horse or primate).

As used herein, dendrimer is a unimolecular assembly comprising thee elements: (i) an initiator core, (ii) interior layers (which correspond to the generation G) consisting of repeating units, radially attached to the initiator core and (iii) an exterior surface.

PAMAM: Polyamidoamine

PAMAM dendrimer: a polyamidoamine dendrimer, wherein the initiator core is an ehtylenediamine core, and the branched units (layers) are constructed based on methyl acrylate and ethylendiamine.

PAMAM Gn or Gn PAMAM: A polyamidoamine dendrimer of n^{th} generation. For example, G4 PAMAM is a 4^{th} generation polyamidoamine dendrimer.

Tos: CH₃C₆H₄SO₂ or Tosyl group.

Cbz: carboxybenzyl group.

### BRIEF DESCRIPTION OF THE INVENTION

The present invention considers derivatized nanomolecules with proven effectiveness to bind to actinides, more specifically uranium, during in vivo assays, showing a reduction in at least kidney damage due to exposition to uranium.

### DETAILED DESCRIPTION OF THE INVENTION

As previously stated, the present invention corresponds to different derivatized nanomolecules with proven effectiveness for in vivo removal of heavy metals or actinides, more specifically uranium or depleted uranium.

More particularly, the derivatized nanomolecules of the present invention correspond to derivatized polyamidoamine (PAMAM) dendrimers of different generations.

In a more particular embodiment of the invention, the derivatized PAMAM dendrimers are of generations 2nd, 3rd, 4th, 5th, or 6th. In an alternative embodiment, the derivatized nanomolecules are half-generation polyamidoamine (PAMAM) dendrimers, i.e. 0.5, 1.5, 2.5, 3.5, 4.5 generation dendrimers.

In a more preferred embodiment, the derivatized nanomolecules of the invention are polyamidoamine (PAMAM) dendrimers of generations 4 and 5.

In a particular embodiment of the invention, the derivatization of the nanomolecules comprises a derivatization group.

In a more specific embodiment of the invention, the derivatization group corresponds to an amino acid conjugated with a terminal functional group.

In a more particular embodiment of the invention, the aminoacid is a basic aminoacid, such as histidine, lysine, or arginine.

In a particular embodiment of the invention, the terminal functional group conjugated to the amino acid is a carbamate or a tosyl group.

In a more specific embodiment, the carbamate group is a carboxybenzyl group.

### Pharmaceutical Compositions

The present invention comprises pharmaceutical compositions comprising at least one of the dendrimers considered in the present invention, a suitable solvate, suitable salts, and/or excipients.

The molecules of the invention (i.e. active ingredient) can be included in different dosage forms, such as for example, and with no intention of limiting the scope of the invention, oral dosage forms, such as pills, films, tablets, capsules, dragees, pastes, powders, liquid solutions or suspensions; parenteral dosage forms, suitable for intradermal, intramuscular, intraosseous, intraperitoneal, intravenous, subcutaneous, or intrathecal administration; topical dosage forms such as creams, gels, liniments, balms, lotions, ointments, skin patches.

In a particular embodiment of the invention, the dosage for reaching an effective removal of heavy metals or actinides, more particularly uranium or depleted uranium, from a patient, correspond to a dosage of at least 5 mg/kg, at least 7 mg/kg, at least 10 mg/kg, at least 15 mg/kg, at least 20 mg/kg, at least 25 mg/kg, at least 30 mg/kg.

### Methods

The present invention further encompasses the use of the molecules described herein in the treatment or alleviation of damages that may be caused by heavy metal toxicity, actinide derived toxicity, and more particularly, toxicity caused by exposure to uranium or depleted uranium.

In one aspect of the invention, a pharmaceutical composition comprising the molecules of the invention is injected in a patient in need thereof.

In one embodiment of the invention, the injection or application of the molecules of the invention for providing in vivo chelating of heavy metals or actinides, more specifically uranium or depleted uranium, from a patient in need thereof is achieved when reaching a concentration of at least 1 μM, more preferentially at least 3 μM, and even more preferentially at least 5 μM. Other higher concentrations are also considered to be in the scope of the present invention, such as up to 10 μM, or up to 15 μM.

In a further embodiment, the molecules of the invention can be immobilized in a suitable matrix for extracorporeal removal of heavy metals, such as for example through dialysis, or for ex vivo removal of the heavy metals.

A further embodiment of the invention considers the use of the disclosed compounds immobilized in a suitable matrix for removal of actinides or heavy metals from liquid solutions. For example, the compounds of the invention can be immobilized in a cartridge for the treatment of a body of water, wherein the water is passed through the matrix and the heavy metals, actinides, or more specifically uranium or depleted uranium, are bound to the compounds of the invention and retained in the matrix.

Another embodiment of the invention considers the application of the molecules described herein to contaminated environment, i.e. in situ treatment or removal of heavy metals, more particularly actinides, and more specifically uranium or depleted uranium.

### EXAMPLES

### Example 1

### Dendrimer Synthesis

PAMAM G4-Arginine-Tos-OH (G4-Arg-Tos). Conjugation of PAMAM-G4 dendrimer (Dendritech, Midland, Mich.) with Boc-Arginine (Tos)-OH (Sigma Aldrich Co. Saint-Louis, Mo.) was carried out by a condensation between the carboxyl group of the Arginine and the primary amino group of PAMAM. Thus, 64 mg (0.208 mmoles) of Boc-arginine (Tos)-OH reacted with 150 mg (2.9 mmoles) of EDC and 150 mg of HOBt in a mixture of 2.7 mL of dry DMF and 1.0 mL of dry DMSO under a nitrogen atmosphere for 1 hr. The reaction mixture was added dropwise to a solution of 40 mg (1.38×10⁻³ mmoles) of PAMAM G4 in 3 mL of water. The reaction mixture was vigorously stirred for 72 hrs. The functionalized dendrimer was purified through dialysis membranes with a cut-off of 500 Da to take off the excess of the amino acids. Then, the product was lyophilized and the amount of the PAMAM G4-Boc-Arginine (Tos)-OH obtained was 55 mg. Finally, a solution of HCl/dioxane (4 mL, 4 M) in a 25 mL round-bottom flask equipped with a magnetic stirrer was cooled by an ice-water bath under nitrogen and PAMAM G4-Boc-Arginine (Tos)-OH (0.2 mmol) was added in one portion with stirring. The ice-bath was removed and the mixture was kept stirred for 1 hr., thing layer chromatography (TLC) indicated that the reaction was completed. The reaction mixture was condensed by rotary evaporation under high vacuum at room temperature. The residue was then washed with dry ethyl ether and collected by filtration (for oil products, a simple decantation was used instead). Yield: >95% of PAMAM G4-Arg-Tos

PAMAM G4-Lysine-Fmoc-Cbz (G4-Lys-Fmoc-Cbz). Conjugation of PAMAM-G4 dendrimer with Fmoc-Cbz-Lysine was carried out according to the previously reported method (Geraldo et al., Pisal et al., 2008). Briefly, 123 mg (0.24 mmoles) of Fmoc-Cbz-lysine reacted with 38 mg (0.24 mmoles) of EDC and 33 mg of HOBt (0.24 mmoles) in a mixture of 2.9 mL of dry DMF and 1.0 mL of dry DMSO under a nitrogen atmosphere for 1 hour. The reaction mixture was added dropwise to a solution of 50 mg (3.5×10⁻³ mmoles) of PAMAM G4 in 5 mL of water. The reaction mixture was vigorously stirred for 72 hrs. The functionalized dendrimer was purified through dialysis membranes with a cut-off of 500 Da to take off the excess of amino acids. After lyophilization, the amount of the PAMAM-Lys-Fmoc was 73 mg.

PAMAM G4-Lysine-Cbz-OH (G4-Lys-Cbz). Conjugation of PAMAM-G4 dendrimer with Boc-Lys-Cbz-OH was carried out according to the previously reported method (Geraldo et al., Pisal et al., 2008). Briefly, 56 mg (0.208 mmoles) of Boc-Lys-Cbz-OH reacted with 150 mg (2.9 mmoles) of EDC and 150 mg of HOBt in a mixture of 2.7 mL of dry DMF and 1.0 mL of dry DMSO under a nitrogen atmosphere for 1 hr. The reaction mixture was added dropwise to a solution of 30 mg (2.1×10⁻³ mmoles) of PAMAM in 3 mL of water. The reaction mixture was vigorously stirred for 72 hrs. The functionalized dendrimer was purified through dialysis membranes with a cut-off of 500 Da to take off the excess of the amino acids. After the lyophilization, the amount of the PAMAM G4-Boc-Lysine-Cbz-OH obtained was 42 mg. Finally, a solution of HCl/dioxane (4 mL, 4 M) in a 25 mL round-bottom flask equipped with a magnetic stirrer was cooled by an ice-water bath under nitrogen, and PAMAM G4-Boc-Lysine-Cbz-OH (0.2 mmol) was added in one portion with stirring. The ice-bath was removed and the mixture was kept stirred for 1 hr., TLC indicated that the reaction was completed. The reaction mixture was condensed by rotary evaporation under high vacuum at room temperature. The residue was then washed with dry ethyl ether and collected by filtration (for oil products, a simple decantation was used instead). Yield: >95% of PAMAM G4-Lys-Cbz.

PAMAM-G4-Folate (G5-FA). Conjugation of PAMAM-G5 dendrimer (Dendritech, Midland, Mich.) with folic acid (FA) (Sigma) was carried out by a condensation between the γ-carboxyl group of FA and the primary amino group of PAMAM. Thus, 104 mg (0.23 mmoles) of FA reacted with 150 mg (1.28 mmoles) of 1-[3-(dimethylamino) propyl]-3-ethylcarbodi-imide HCl (EDC) (Sigma Aldrich Co. Saint-Louis, Mo.) and 150 mg of N-hydroxybenzotriazole (HOBt) (Sigma Aldrich Co. Saint-Louis, Mo.), in a mixture of 2.7 mL of dry N-dimethylformamide (DMF) (Sigma Aldrich Co. Saint-Louis, Mo.), and 1.0 mL of dry dimethyl sulfoxide (DMSO), (Sigma Aldrich Co. Saint-Louis, Mo.) under a nitrogen atmosphere for 1 hour. The reaction mixture was added dropwise to a solution of 40 mg (1.38×10⁻³ mmoles) of PAMAM G5 in 3 mL of water. The reaction mixture was vigorously stirred for 72 hrs. The functionalized dendrimer was purified through dialysis (using water) membranes with a cut-off of 500 Da (Spectrum laboratories, Inc., Rancho Dominguez, Calif.) to take off the excess of folate. After the lyophilization (Freezone 6, Labconco, USA), the amount of the PAMAM G5-FA obtained was 52 mg.

PAMAM-G5-Coumarin (G5-Cou). Conjugation of PAMAM-G5 dendrimer with Coumarin-3-carboxylic acid (Cou) (Sigma Aldrich Co. Saint-Louis, Mo.) was carried out by a condensation between the carboxyl group of Cou and the primary amino group of PAMAM. Thus, 39.5 mg (0.208 mmoles) of Cou reacted with 32 mg (0.208 mmoles) of EDC in a mixture of 2.7 mL of dry DMF and 1.0 mL of dry DMSO under a nitrogen atmosphere for 1 hr. The reaction mixture was added dropwise to a solution of 40 mg (1.38×10⁻³ mmoles) of PAMAM G5 in 3 mL of water. The reaction mixture was vigorously stirred for 72 hrs. The functionalized dendrimer was purified through dialysis (using water) membranes with a cut-off of 500 Da to take off the excess of Cou. After the lyophilization, the amount of the PAMAM G5-Cou obtained was 55 mg.

MALDI analysis. To confirm the molecular weight of surface modified dendrimers, mass spectral analysis of the dendrimers was performed on a MALDI-TOF (matrix assisted laser desorption/ionization-time of flight) (Bruker, USA) with a pulsed nitrogen laser (337 nm), operating in positive ion reflector mode, using 19 kV acceleration voltage and a matrix of 2,5 dihydroxybenzoic acid (DHB) (Sigma Aldrich Co. Saint-Louis, Mo.).

### Example 2

### Affinity of Derivatized Dendrimers to Uranium

Affinity Assay. For each dendrimer, batch experiments were carried out to determine the extent of binding (ExtB) and fractional binding (FBin) of U(VI) in aqueous solutions. The U(VI) concentration was kept constant at 10 ppm (3.7×10⁻⁵ M) in all experiments. Aliquots of U(VI), dendrimer stock solution or deionized water were added to each tube to prepare 3 mL solutions with given U(VI)-dendrimer molar ratio. The sealed centrifuge tubes were mixed on a rotary shaker for 120 min. Then, the solution was subsequently withdrawn from each equilibrated tube and transferred into a Millipore Centricon filter with a molecular weight cut-off of 5000 Dalton. The filters were centrifuged for 20 min at 3000 rpm to separate de uranyl-laden dendrimers from the aqueous solutions. The concentrations of uranyl in each centrifuge tube (U₀) were measured by fluorescence spectrophotometry. Following (Gu et al., 2005), each sample was diluted 2-fold with a 10% wt of a H₃PO₄ solution. The complexation of U(VI) ions with phosphoric acid causes a large enhancement of their fluorescence emission intensity. This provides the basis of a uranyl assay method with a detection limit of aprox. 40 ppb (Gu et al., 2005, Diallo et al., 2008).

All fluorescence emission spectra were collected on a steady-state RF-5301pc Spectrofluorophotometer (Shimadzu) using an excitation wavelenght of 280 nm. The emission spectra were recorded between 480 and 545 nm. The intensity of the emission peak at 508 nm was used to develop the U(VI) calibration curve.

The concentration of uranyl bound to a dendrimer (U_{b}) (mol/L) was expressed as follows:

U_{b}=U_{c}−U₀

Where U_{c} is the uranyl concentration of the control (uranyl without dendrimer) after the incubation and centrifugation. The extent of binding (ExtB) (moles of U(VI) bound per mole of dendrimer), the concentration of dendrimer (Cden) in solution (mol/L) and the fractional binding (FBin) were expressed as follows:

ExtB=U_{b}/Cden

Cden=m_{d}/Vₛ*M_{wd}

FBin=100*(U_{b}/U₀)

where m_{d} (g) is the mass of dendrimer in solution, Vₛ (L) is the solution volume and M_{wd} (g/mol) is the molar mass of the dendrimer.

### Example 3

### Red Blood Cells (RBCs) Interactions

Sample Collection. Blood from healthy donors was extracted and anticoagulated with heparine tubes (BD Vacutainer, Becton Dickinson). Erythrocytes were separated from the plasma and leucocytes by centrifugation (1500 rpm, 5 min) at 4° C. and washed three times with saline (NaCl 0.9%) (Sigma Aldrich Co. Saint-Louis, Mo.).

All the protocols were authorized by the ethic committee of the University of Talca in accordance with the Declaration of Helsinki (approved by the 18th World Medical Assembly in Helsinki, Finland, in 1964).

Assay of Red Blood Cells Lysis. The hemolysis assay was performed according to the method of Duncan et al., (Duncan R., 1992). Briefly, washed RBCs at 2% were incubated at room temperature with a concentration of 4 μM of the selected dendrimer. After 2 hours of incubation, samples were centrifuged at 2000 rpm for 10 minutes and the absorbance of the supernatant was measured at 550 nm (Clima Plus, RAL S. A, Barcelona). Hemolysis was expressed as percentage of released hemoglobin, used as control (100% of hemoglobin released) a solution of RBCs incubated with Triton X-100 (0.2% v/v, Sigma Aldrich Co. Saint-Louis, Mo.).

### Example 4

### In Vivo Acute Toxicity Assay

Animals. A total of 48 male C57BL/6 mice weighing 25-30 g purchased from the Instituto de Salud Pública (ISP), were used. Animal housing and experiment protocols were approved by the University of Talca in adherence to Conicyt experimental animal administrative regulations. All groups were maintained at a constant temperature (22±1° C.) on a 12-h light/12-h dark cycle (lights-on at 08:00 a.m.) and had free access to food and water.

Animal experimental design. Eight groups of study were designed; the first group (n=6) was designated as the control group (saline, ip), the second group (n=6) for uranyl acute intoxication (UO₂(CH₃COO)₂.2H₂O, 5 mg/kg of uranium, ip), the third group (n=6) for G4-Lys-Fmoc-Cbz (16 mg/kg, ip), the fourth group (n=6) for G5-Cou (40 mg/kg, ip), the fifth group (n=6) for ethylenediaminetetraacetic acid (EDTA, 75 mg/kg, ip), the sixth group (n=6) for G4-Lys-Fmoc-Cbz (16 mg/kg, ip) immediately after U(VI) administration (5 mg/kg, ip), the seventh group (n=6) for G5-Cou (40 mg/kg) immediately after U(VI) administration (5 mg/kg, ip) and the eighth group (n=6) for EDTA (75 mg/kg, ip) immediately after U(VI) administration (5 mg/kg, ip). Animals were euthanized 48 h later and blood samples were collected from the heart for the assessment of serum urea, creatinine, Lactate Deshidrogenase (LDH), Uric Acid and Calcium

Examination of nephrotoxicity. Mice were anaesthetized with sodium pentobarbitone and blood samples were collected from the heart and the kidneys were removed. Serum levels of urea, creatinine, LDH, Uric Acid and Calcium were determined using commercially available kits according to the protocols provided by Valtek (Valtek Diagnostic, Nuñoa, Chile). Left kidneys were fixed in 10% neutral buffered formaldehyde, embedded in paraffin wax and automatically processed. Sections (3 μm in thickness) of the embedded tissue were stained with hematoxylin-eosin for observation under light microscope.

Statistical analysis. The in vitro data were obtained from at least three independent experiments with three replicates, and all data are expressed as mean±SD. Experimental groups were compared using a one-way analysis of variance (ANOVA) followed by Scheffé's test when the data were normally distributed and by the Kruskal-Wallis test when they were not normally distributed. p-Values <0.05 were considered significant. Statistical tests were performed using GraphPad Prism 5 software, version 5.0 for Mac OS X.

### Results

### Dendrimers Synthesis

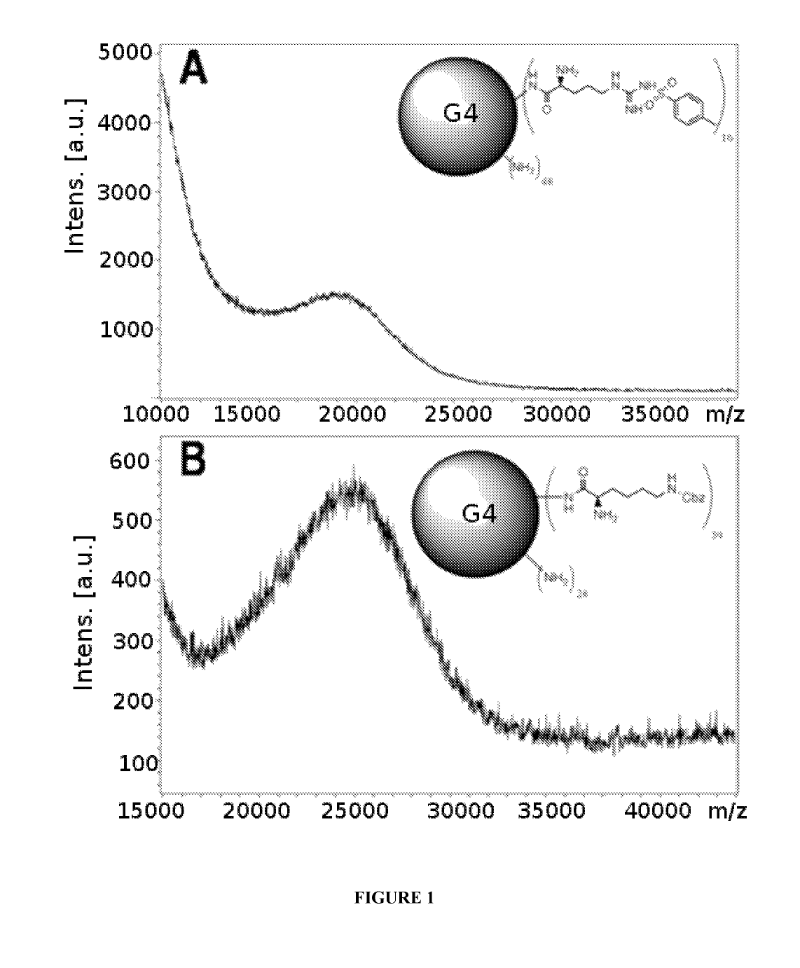

Lysine, arginine and folic acid were coupled to the amine terminals of the PAMAM G4 dendrimers by EDC and HOBt coupling reaction (Geraldo et al.). A higher molar ratio of 1:70 was used to get all the 64-amine groups of the PAMAM G4 dendrimer conjugated. Nevertheless, only 16 Arg-Tos, 39 Lys-Cbz, 2 Lys-Fmoc-Cbz and 23 FA molecules were found attached to the PAMAM G4 dendrimer, respectively. The difference in the number of molecules coupled to PAMAM G4 was due perhaps to the steric hindrance caused by the size of the molecules. To avoid dimerization and to increase the degree of conjugation, Boc synthesis protocol was used. The modification of the dendrimers surface were characterized by MALDI-TOF spectroscopic analysis (FIG. 1).

Coumarin-3-carboxylic acid was coupled to the amine terminals of the PAMAM G5 dendrimers by EDC and HOBt coupling reaction (Geraldo et al.). A higher molar ratio of 1:140 was used to get all the 128-amine groups of the PAMAM G5 dendrimer conjugated. However, only 64 Cou molecules were found attached to the PAMAM G5 dendrimer. The difference in the number of molecules coupled to PAMAM G5 was due perhaps to steric hindrance caused by the size of the coumarin. All dendrimers synthesized had a yield over 90%, the characteristics of each of them are listed in Table 1.

TABLE 1
Characterization of PAMAM G4 and G5 derivatives synthesized.

| | Molecular | Surface | N° Chemical | |
|---|---|---|---|---|
| Dendrimer | weight | groups | groups | Yield |
| G4 | 14215 | Amine | 64 | |
| G5 | 28826 | Amine | 128 | |
| G4-Arg-Tos | 19500 | Tos-Arginine | 16 | 95% |
| G4-FA | 37289 | Folic Acid | 23 | 93% |
| G4-Lys-Fmoc-Cbz | 15100 | Cbz/Fmoc-Lysine | 2 | 90% |
| G4-Lys-Cbz | 23851 | Cbz-Lysine | 39 | 92% |
| G5-Cou | 41566 | Coumarine | 67 | 94% |

### Effects of Derivatized PAMAM on Uranyl Trapping

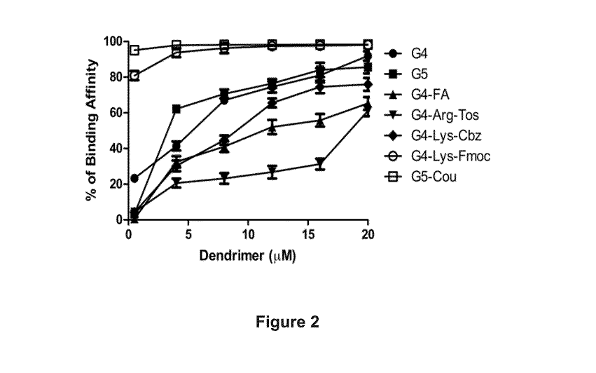

Each of the dendrimers synthesized was incubated with a solution of U(VI) (10 ppm) for 2 hr., at the end of the incubation time, the solution was centrifuged and the concentration of the uranyl unbound to the dendrimer was quantified by spectrofluorimetry. In this regard, the commercial G4 and G5 dendrimers showed a FBin rate of 93% and 86%, respectively, at a dendrimer concentration of 20 μM. The FBin was considerably reduced by lowering the concentration of the dendrimer in the solution (4 μM), showing a binding percentage of 41% and 63% respectively (FIG. 2). For its part, dendrimers G4-Arg-Tos, G4-FA and G4-Lys-Cbz, at a concentration of 20 μM, showed a lower FBin that the commercial dendrimers, with percentages of 62, 65 and 77%, respectively. However, dendrimers G4-Lys-Fmoc-Cbz and G5-Cou showed high percentage of trapping, reaching about 90% of FBin at a concentration of 4 μM. Also, the dendrimers G4-Lys-Fmoc-Cbz and G5-Cou, showed the higher extent of binding (Table 2), with 35 mol of U(VI) bound per mol of each dendrimer.

TABLE 2
Affinity of PAMAM dendrimers synthesized for uranyl

| | | | G4-Arg- | G4-Lys- | G4-Lys- | | |
|---|---|---|---|---|---|---|---|
| | G4 | G5 | Tos | Fmoc-Cbz | Cbz | G4-FA | G5-Cou |
| Cden | 5.0E−07 | 4.0E−06 | 5.0E−07 | 5.0E−07 | 5.0E−07 | 4.0E−06 | 5.0E07 |
| U_{b} | 3.5E−06 | 1.0E−05 | 1.0E−06 | 1.8E−05 | 1.1E−06 | 6.8E−06 | 1.5E−05 |
| FBin (%) | 23.8 | 62.6 | 4.6 | 81.7 | 4.6 | 33.7 | 95.4 |
| ExtB | 7.1 | 2.6 | 2 | 35 | 2.2 | 1.7 | 34 |

Cden: concentration of the dendrimer in solution (mol/L), U_{b}: dendrimer bound to uranyl (mol/L), FBin (%): percentage of fraction bound; ExtB: degree of union (U mol/mol dendrimer).

### Interaction of Derivatized Dendrimers on Red Blood Cells (RBCs)

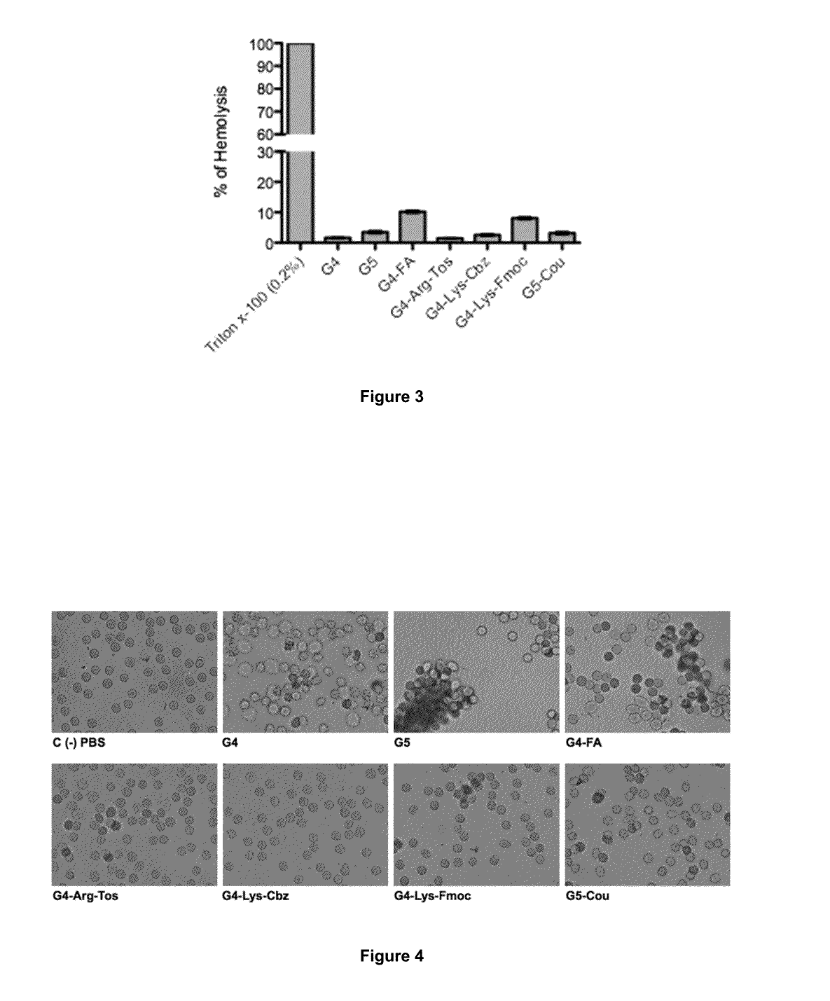

The FIG. 3 shows the hemolysis of RBCs expressed as percentage of released hemoglobin compared to the positive control Triton X-100 (0.2%, v/v). These findings indicate that PAMAM dendrimers G4-FA and G4-Lys-Fmoc-Cbz (final concentration of 4 μM in saline) have the highest percentage of hemolysis (10.2% and 8.1%, respectively). Moreover, FIG. 4 shows the differences between negative control and the different dendrimers tested. It can be observed that the commercial dendrimers PAMAM G4 and G5 cause intense agglutination of RBCs, whereas PAMAM G4 derivatives have no greater interaction with the RBCs, however, the PAMAM G5 derivatives afforded moderate agglutination of the RBCs.

### Example 5

### In Vivo Effects of the Selected Dendrimers

As shown in Table 3, after U(VI) administration of 5 mg/kg, the concentration of urea and uric acid increased significantly (p<0.001) in all groups exposed to U (VI), compared to control (saline), at the same time, none of the used dendrimers or EDTA caused changes in these biochemical parameters.

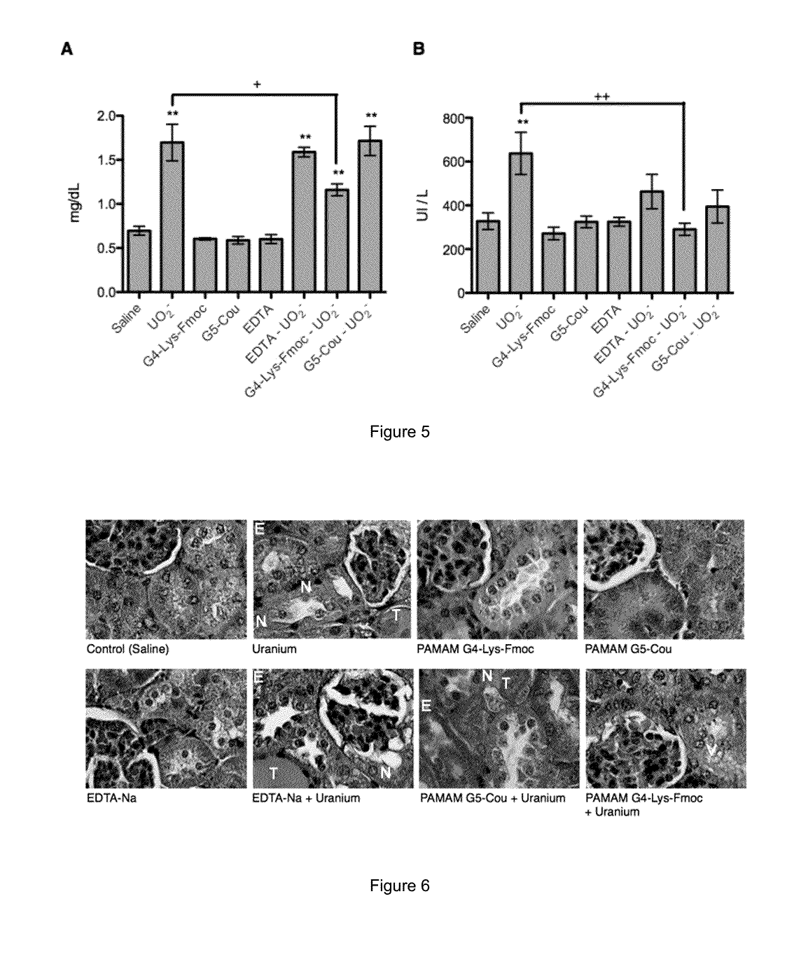

On the other hand, when analyzing concentration of creatinine, an increase in the levels of all groups exposed to U(VI) (p<0.001) is observed, nevertheless, the concentration of creatinine in G4-Lys-Fmoc-Cbz-UO₂⁻ group was significantly lower (p<0.05) than positive control (UO₂⁻) (1.2±0.17 versus 1.7±0.5, respectively), said effect was not observed in group G5-Cou-UO₂⁻ (p>0.05, FIG. 5a). Regarding measurement of LDH, a wide spectre toxicity indicator, it was observed that U(VI) induced a significant increase of the enzyme (p<0.05), nevertheless, the use of different dendrimers and EDTA decreases said generalized toxicity, even more, G4-Lys-Fmoc-Cbz dendrimer was able to keep the levels in normal range obtaining a significant difference to the positive control (UO₂⁻) and the group G4-Lys-Fmoc-Cbz-UO₂⁻ (p<0.001), as shown in FIG. 5b.

TABLE 3
Urea, uric acid, and calcium concentration in different studied groups.

| | | | | | | | G4-Lys- | |
|---|---|---|---|---|---|---|---|---|
| | | | | | | EDTA- | Fmoc-Cbz | G5-Cou- |
| | Saline | UO₂⁻ | G4-Lys | G5-Cou | EDTA | UO₂⁻ | UO₂⁻ | UO₂⁻ |
| Urea | 53.3 ± 2.2 | 226.3 ± 39.4** | 46.3 ± 4.1 | 43.5 ± 2.2 | 90.0 ± 17.7 | 192.5 ± 4.4** | 166.3 ± 20.9** | 202.3 ± 31.0** |
| (mg/dL) | | | | | | | | |
| Uric Acid | 0.77 ± 0.15 | 2.5 ± 0.39** | 1.4 ± 0.21 | 1.4 ± 0.36 | 2.2 ± 0.40 | 3.2 ± 0.35** | 3.3 ± 0.16** | 2.8 ± 0.35** |
| (mg/dL) | | | | | | | | |
| Calcium | 7.0 ± 0.23 | 8.8 ± 0.57* | 7.3 ± 0.42 | 6.9 ± 0.26 | 6.8 ± 0.39 | 7.7 ± 0.38 | 8.4 ± 0.31 | 7.9 ± 0.36 |
| (mg/dL) | | | | | | | | |

*p < 0.05,
**p < 0.001 compared to the saline control.

Using hematoxylin-eosin staining of kidneys obtained from mice from different studied groups, optical microscopy allowed to see that the control group (saline), preserves the structure of renal tubules and glomeruli, and on the other hand, the positive control (UO₂⁻), showed irreversible damage with necrosis, also showing karyolysis and total eosinophilia of cytoplasm together with tubule dilation with atrophy or “thyroidization”. PAMAM G4-Lys-Fmoc-Cbz and G5-Cou controls, together with EDTA-Na did not show differences compared to the positive control, standing out that low interaction with red cells and hemolysis, they also did not cause damage or toxicity (reflected as LDH activity) in experimental animals. Nevertheless, when using these dendrimers together with U(VI), in the case of acute intoxication (5 mg/kg), only PAMAM G4-Lys-Fmoc-Cbz dendrimer was able to protect the kidney from damage produced by this metal (FIG. 6), since the case of PAMAM G5-Cou dendrimer+UO₂⁻ and EDTA-Na+UO₂⁻, irreversible damage with necrosis and in some cases karyolysis and total eosinophilia, and thyroidization was observed, although, when observing the tissue of the group treated with PAMAM G4-Lys-Fmoc-Cbz+UO₂⁻, some damage was observed but it was reversible with a slight increase of eosinophilia in cytoplasm and luminal surface vesiculations.

### DISCUSSION AND CONCLUSION

The global distribution of uranium (U) contamination has remained a persistent environmental and human health problem for several decades. U is a naturally occurring radioactive heavy metal derived from the earth's crust and is composed of three naturally occurring isotopes, U²³⁴, U²³⁵, and U²³⁸. DU is a waste product of the U enrichment process wherein the more highly radioactive isotopes, U²³⁴ and U²³⁵, are removed from natural U, leaving a waste material that is largely but not completely “depleted” of these isotopes with higher specific activities. The specific activity of DU is about 60% that of natural U because of this isotopic difference (Army Environmental Policy Institute (AEPI). 1995), but it retains the chemical toxicity of natural U as a heavy metal (The Royal Society., 2001, Society., 2002).

The kidney has long been recognized as the “critical” target of U exposure, i.e., the organ first perturbed (Leggett, 1989), and is considered the primary target organ following both acute and chronic exposures to soluble U compounds (McDiarmid et al., 2001, Parkhurst, 2003). Animal evidence also documents other targets of U exposure, including the bone, reproductive, and central nervous systems (Gilman et al., 1998, McDiarmid et al.). Extensive experience demonstrates that acute and chronic human intoxications with a wide range of metals can be treated with considerable efficiency by the administration of a relevant chelating agent (Flora et al., 2008). Thus, a chelating agent forming a stable complex with a toxic metal may shield the metal ion from biological targets, thereby reducing the toxicity, even at times after administration where mobilization has not yet occurred (Andersen, 1999). On this context we synthesized derivatives of PAMAM G4 and G5 and their chelating properties and hemocompatibility were studied.

In case of commercial dendrimers G4 and G5, these showed a good capacity of trapping the uranyl ion, nevertheless, when incubated with RBCs, these dendrimers caused a high agglutination of these cells. Similar results were obtained by Wang et al. (Wang et al.), when the PAMAM G4 dendrimer was examined as a nanocarrier candidate for gene delivery. Low doses of PAMAM G4 dendrimer (10 nM-10 μM˜141.3 ng/ml-141.3 μg/ml) caused RBC aggregation and shape changes, from echinocytic, spindle-shaped to spherocyte-like forms, and when the concentration increased to 100 μM (˜1.41 mg/ml), PAMAM G4 induced membrane rupture and disintegration (Ziemba et al.).

However, dendrimers G4-Arg-Tos and G4-Lys-Cbz, showed a percentage entrapment of uranyl ion less than 10%. Despite this, both dendrimers showed low toxicity, with a percentage of hemolisys lower than 10%, at a concentration of 4 μM without agglutination of red blood cells, so that, despite not be good candidates as chelating agent, positions itself as good dendrimers for biological applications because it does not affect the RBCs membranes or cause agglutination thereof.

From the series of dendrimers synthesized G4-Lys-Fmoc-Cbz and G5-Cou, had a higher percentage of trapping even that the commercial dendrimers (G4 and G5), with a percentage of trapping of 81.7 and 95.4% each one, at a concentration of 0.5 μM (7.55 μg/mL y 20.78 μg/mL, respectively), determining that each dendrimer (G4-Lys-Fmoc-Cbz and G5-Cou) is capable of fixing 35 mol of uranyl per mol of dendrimer in solution. Also, both dendrimer G4-Lys-Fmoc-Cbz and the G5-Cou, do not cause hemolysis or agglutination of RBCs at a concentration of 4 μM and the uranyl ion trapping continues performing well.

Finally, from the series of dendrimers synthesized, only G4-Lys-Fmoc-Cbz and G5-Cou, at a concentration of 4 μM, have a high capacity of uranyl ion trapping without causing hemolysis or RBCs agglutination, which makes them good candidates for in vivo studies aimed to obtaining a chelating agent which can be used in case of acute poisoning by uranium.

### REFERENCES

Andersen, O (1999) Principles and recent developments in chelation treatment of metal intoxication. Chem Rev 99: 2683-2710
Army Environmental Policy Institute (AEPI). (1995) Health and Environmental Consequences of Depleted Uranium Use in the U S Army. Atlanta, Ga.: AEPI
Barber, D S, Hancock, S K, McNally, A M, Hinckley, J, Binder, E, Zimmerman, K, Ehrich, M F and Jortner, B S (2007) Neurological effects of acute uranium exposure with and without stress. Neurotoxicology 28: 1110-1119
Boas, U and Heegaard, P M (2004) Dendrimers in drug research. Chem Soc Rev 33: 43-63
Bumb, A, Brechbiel, M W and Choyke, P Macromolecular and dendrimer-based magnetic resonance contrast agents. Acta Radiol 51: 751-767
Crooks, R M, Zhao, M, Sun, L, Chechik, V and Yeung, L K (2001) Dendrimer-encapsulated metal nanoparticles: synthesis, characterization, and applications to catalysis. Acc Chem Res 34: 181-190
Diallo, M S, Arasho, W, Johnson, J H, Jr. and Goddard, W A, 3rd (2008) Dendritic chelating agents. 2. U(V I) binding to poly(amidoamine) and poly(propyleneimine) dendrimers in aqueous solutions. Environ Sci Technol 42: 1572-1579
Duncan R. (1992) Progress in Membrane Biotechnology. In: Gomez Fernandez, J. C., Chapman, D., Packer, L. (Eds.). Birkhauser Verlag, Basel, Switzerland.: 253-265
Dutta, T, Jain, N K, McMillan, N A and Parekh, H S Dendrimer nanocarriers as versatile vectors in gene delivery. Nanomedicine 6: 25-34
Flora, S J, Mittal, M and Mehta, A (2008) Heavy metal induced oxidative stress & its possible reversal by chelation therapy. Indian J Med Res 128: 501-523
Flora, S J and Pachauri, V Chelation in metal intoxication. Int J Environ Res Public Health v.7(7); July 2010
Geraldo, D A, Duran-Lara, E F, Aguayo, D, Cachau, R E, Tapia, J, Esparza, R, Yacaman, M J, Gonzalez-Nilo, F D and Santos, L S Supramolecular complexes of quantum dots and a polyamidoamine (PAMAM)-folate derivative for molecular imaging of cancer cells. Anal Bioanal Chem 400: 483-492
Gilman, A P, Moss, M A, Villeneuve, D C, Secours, V E, Yagminas, A P, Tracy, B L, Quinn, J M, Long, G and Valli, V E (1998) Uranyl nitrate: 91-day exposure and recovery studies in the male New Zealand white rabbit. Toxicol Sci 41: 138-151
Gu, B, Ku, Y K and Brown, G M (2005) Sorption and desorption of perchlorate and U(V I) by strong-base anion-exchange resins. Environ Sci Technol 39: 901-907
Hartmann, H M, Monette, F A and Avci, H I (2000) Overview of Toxicity Data and Risk Assessment Methods for Evaluating the Chemical Effects of Depleted Uranium Compounds. Human and Ecological Risk Assessment: An International Journal 6: 851-874
Ilaiyaraja, Ashish Kumar Singha Deb, K. Sivasubramanian, D. Ponraju, B. Venkatraman (2013) Adsorption of uranium from aqueous solution by PAMAM dendron functionalized styrene divinylbenzene, Journal of Hazardous Materials 250-251 (2013) 155-166
Leggett, R W (1989) The behavior and chemical toxicity of U in the kidney: a reassessment. Health Phys 57: 365-383
Mansfield, M L and Klushin, L I (1992) Intrinsic viscosity of model Starburst dendrimers. The Journal of Physical Chemistry 96: 3994-3998
May, L M, Heller, J, Kalinsky, V, Ejnik, J, Cordero, S, Oberbroekling, K J, Long, T T, Meakim, K C, Cruess, D and Lee, A P (2004) Military deployment human exposure assessment: urine total and isotopic uranium sampling results. J Toxicol Environ Health A 67: 697-714
McDiarmid, M A, Albertini, R J, Tucker, J D, Vacek, P M, Carter, E W, Bakhmutsky, M V, Oliver, M S, Engelhardt, S M and Squibb, K S Measures of genotoxicity in Gulf war I veterans exposed to depleted uranium. Environ Mol Mutagen 52: 569-581
McDiarmid, M A, Squibb, K, Engelhardt, S, Oliver, M, Gucer, P, Wilson, P D, Kane, R, Kabat, M, Kaup, B, Anderson, L, Hoover, D, Brown, L and Jacobson-Kram, D (2001) Surveillance of depleted uranium exposed Gulf War veterans: health effects observed in an enlarged “friendly fire” cohort. J Occup Environ Med 43: 991-1000
Newkome G R., Yao Z Q., Baker G R. and Gupta V K. (1985) Cascade molecules: A new approach to micelles. A [27]-arborol. J Org Chem. 50: 2003-2006
Parkhurst, M A (2003) Measuring aerosols generated inside armoured vehicles perforated by depleted uranium ammunition. Radiat Prot Dosimetry 105: 167-170
Pisal, D S, Yellepeddi, V K, Kumar, A, Kaushik, R S, Hildreth, M B, Guan, X and Palakurthi, S (2008) Permeability of surface-modified polyamidoamine (PAMAM) dendrimers across Caco-2 cell monolayers. Int J Pharm 350: 113-121
Pittelkow, M, Nielsen, C B, Broeren, M A, van Dongen, J L, van Genderen, M H, Meijer, E W and Christensen, J B (2005) Molecular recognition: comparative study of a tunable host-guest system by using a fluorescent model system and collision-induced dissociation mass spectrometry on dendrimers. Chemistry 11: 5126-5135
Society., T R (2002) The Health Effects of Depleted Uranium Munitions. Summary. London, U K: Royal Society
Taulan, M, Paquet, F, Argiles, A, Demaille, J and Romey, M C (2006) Comprehensive analysis of the renal transcriptional response to acute uranyl nitrate exposure. BMC Genomics 7: 2
The Royal Society. (2001) The Health Hazards of Depleted Uranium Munitions Part I. Policy Document 6/01. London, U K: Royal Society
Tomalia, D A, Baker, H, Dewald, J, Hall, M, Kallos, G, Martin, S, Roeck, J, Ryder, J and Smith, P (1985) A New Class of Polymers: Starburst-Dendritic Macromolecules. Polymer Journal 17: 117-132
Wang, P, Zhao, X H, Wang, Z Y, Meng, M, Li, X and Ning, Q Generation 4 polyamidoamine dendrimers is a novel candidate of nano-carrier for gene delivery agents in breast cancer treatment. Cancer Lett 298: 34-49
Ziemba, B, Matuszko, G, Bryszewska, M and Klajnert, B Influence of dendrimers on red blood cells. Cell Mol Biol Lett 17: 21-35

1. Derivatized dendrimer with low citotoxicity for in vivo, ex vivo, in vitro or in situ chelation of heavy metals or actinides, comprising a polyamidoamine (PAMAM) dendrimers derivatized with an amino acid and a terminal functional group.

2. The derivatized dendrimer according to claim 1, wherein the amino acid is a basic amino acid.

3. The derivatized dendrimer according to claim 1, wherein the amino acid is selected among histidine, lysine, or arginine.

4. The derivatized dendrimer according to claim 1, wherein the terminal functional group is a carbamate.

5. The derivatized dendrimer according to claim 4, wherein the terminal functional group is a carboxybenzyl group.

6. The derivatized dendrimer according to claim 1, wherein the terminal functional group is a tosyl group.

7. The derivatized dendrimer according to claim 1, wherein the derivatized PAMAM dendrimers are of generations 2nd, 3rd, 4th, 5th, or 6th.

8. The derivatized dendrimer according to claim 1, wherein the derivatized PAMAM dendrimers are of generations 0.5, 1.5, 2.5, 3.5, 4.5.

9. Pharmaceutical composition for treating a blood clotting disorder comprising at least one derivatized dendrimer according to claim 1, a suitable solvate, suitable salts, and or excipients.

10. Method for treating exposure of a subject to a heavy metal or acitinide, comprising the administration of a pharmaceutical composition comprising at least one of molecules according to claim 1, to a patient in need thereof.

11. Method for removal of heavy metals or actinides from a liquid solution, comprising the use of immobilized molecules according to claim 1 to a suitable matrix, and exposing the liquid solution to said matrix containing said immobilized molecules.
